SEQUENCE LISTING (6 sequences)

SEQ ID NO: 1 - protein, 8 aa, Homo sapiens
GYTFTNYY

SEQ ID NO: 2 - protein, 8 aa, Homo sapiens
INPSNGGT

SEQ ID NO: 3 - protein, 13 aa, Homo sapiens
TRRDYNYDGGFDY

SEQ ID NO: 4 - protein, 9 aa, Homo sapiens
KSVSTSGFN

SEQ ID NO: 5 - protein, 3 aa, Homo sapiens
LAS

SEQ ID NO: 6 - protein, 9 aa, Homo sapiens
QHGRELPLT

(19) United States
(12) Patent Application Publication
(10) Pub. No.: US 2020/0046769 A1
(43) Pub. Date: Feb. 13, 2020

(54) DUAL FUNCTION ENGINEERED T CELLS WITH HPV E6 SPECIFICITY AND PD-1 BLOCKADE

(71) Applicant: TCRCURE BIOPHARMA CORP., CHAPEL HILL, NC (US)

(72) Inventors: Pin Wang, Los Angeles, CA (US); Si Li, Alhambra, CA (US)

(21) Appl. No.: 16/537,557

(22) Filed: Aug. 10, 2019

Related U.S. Application Data
(60) Provisional application No. 62/717,787, filed on Aug. 11, 2018.
(60) Provisional application No. 62/731,329, filed on Sep. 14, 2018.

(51) Int. Cl. A61K 35/17; C07K 16/28; C12N 15/86

(52) U.S. Cl. CPC A61K 35/17; A61P 35/00; C12N 15/86; C07K 16/2818

(57) ABSTRACT

The present invention generally relates to engineered cells and compositions thereof, particularly, T cells comprising genetically engineered T Cell receptors (TCRs) and checkpoint inhibitors (CPIs). Methods for using the compositions to treat cancer are also disclosed herein. Genetically engineered T cells that recognize tumor antigen HPV E6 and simultaneously secrete a single-chain antibody that blocks Programmed Cell Death Protein 1 (PD-1). Also provided is an immunotherapy for HPV E6 expression related cancers.

### CROSS REFERENCE TO RELATED APPLICATIONS

This application claims priority to U.S. Provisional Application No. 62/717,787, filed Aug. 11, 2018, and U.S. Provisional Application No. 62/731,329, filed Sep. 14, 2018, the disclosures of both of which are incorporated herein by reference in their entireties.

### TECHNICAL FIELD

The present invention generally relates to engineered cells and compositions thereof, particularly, T cells comprising genetically engineered T Cell receptors (TCRs) and checkpoint inhibitors (CPIs). Methods for using the compositions to treat cancer are also disclosed herein.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The primary cause of some cancer types such as, for example uterine cervical cancer, is human papilloma virus (HPV) infection. Despite advancement in treatments such as chemotherapy, the prognosis for many cancers, including HPV associated cancers, may be poor. Accordingly, there exists an unmet need for additional treatment for cancer, particularly HPV-associated cancers.

The HPV16 is the subtype of HPV that is most commonly associated with malignancy. Without being bound to a particular theory or mechanism, HPV16 is believed to cause cancer at least partly through the actions of the onco-protein E6, which deregulates cell cycle control. HPV16 E6 is constitutively expressed in cancer cells and is not expressed by normal, uninfected human tissues. HPV16E6 is expressed in a variety of human cancers including, but not limited to, cancer of the uterine cervix, oropharynx, anus, anal canal, anorectum, vagina, vulva, and penis.

The T cell receptor may have antigenic specificity for any HPV16 E6 protein. Adoptive cell transfer (ACT), as a modality of immunotherapy for cancer, has demonstrated remarkable success in treating hematologic malignancies and malignant melanoma. An especially effective form of ACT, which uses gene-modified T cells expressing a chimeric antigen receptor (CAR) to specifically target tumor-associated-antigen (TAA), such as CD19 and GD2, has displayed encouraging results in clinical trials for treating such diseases as B cell malignancies and neuroblastoma.

Unlike naturally occurring T cell receptors (TCRs), CARs are artificial receptor consisting of an extracellular antigen recognition domain fused with intracellular T cell signaling and costimulatory domains. CARs can directly and selectively recognize cell surface TAAs in a major histocompatibility class (MHC)-independent manner. Despite the documented success of CAR T cell therapy in patients with hematologic malignancies, only modest responses have been observed in solid tumors. This can be attributed, in part, to the establishment of an immunosuppressive microenvironment in solid tumors. Such milieu involves the upregulation of several intrinsic inhibitory pathways mediated by increased expression of inhibitory receptors (IRs) in T cells reacting with their cognate ligands within the tumor.

So far, several IRs have been characterized in T cells, such as CTLA-4, T cell Ig mucin-3 (TIM-3), lymphocyte-activation gene 3 (LAG-3), and programmed death-1 (PD-1). These molecules are upregulated following sustained activation of T cells in chronic diseases and cancer, and they promote T cell dysfunction and exhaustion, thus resulting in escape of tumor from immune surveillance. Unlike other IRs, PD-1 is upregulated shortly after T cell activation, which in turn inhibits T cell effector function via interacting with its two ligands, PD-L1 or PD-L2. The PD-L1 is constitutively expressed on T cells, B cells, macrophages, and dendritic cells (DCs). It is also shown to be abundantly expressed in a wide variety of solid tumors. In contrast, the expression of PD-L1 in normal tissues is undetectable. As a consequence of its critical role in immunosuppression, PD-1 has been the focus of recent research, aiming to neutralize its negative effect on T cells and enhance antitumor responses. Clinical studies have demonstrated that PD-1 blockade significantly enhanced tumor regression in colon, renal and lung cancers and melanoma.

### SUMMARY OF THE INVENTION

The present invention provides an engineered T cell, comprising: a nucleic acid encoding (a) genetically engineered antigen receptor that specifically binds to an antigen from HPV; and (b) an inhibitory protein that reduces the function, or is capable of effecting reduction of the expression of inhibitory receptors (IRs) on tumors, such as tumor-infiltrating lymphocytes. These engineered T cells demonstrate stronger anti-tumor response and reduced T cell exhaustion.

In an aspect of the invention, the genetically engineered antigen receptor is a T cell receptor and the inhibitory protein blocks Programmed Cell Death Protein 1 (PD-1), wherein the protein is a single chain antibody (scFv).

The anti-PD-1 scFv antibody of the present invention comprises the following motif sequences: a heavy chain CDR1 comprising amino acids having the sequence set forth in SEQ ID NO:1; a heavy chain CDR2 comprising amino acids having the sequence set forth in SEQ ID NO:2; a heavy chain CDR3 comprising amino acids having the sequence set forth in SEQ ID NO:3; a light chain CDR1 comprising amino acids having the sequence set forth in SEQ ID NO:4; a light chain CDR2 comprising amino acids having the sequence set forth in SEQ ID NO:5; and a light chain CDR3 comprising amino acids having the sequence set forth in SEQ ID NO:6.

In an aspect of the invention, the inhibitory nucleic acid molecule comprises a sequence complementary to a PD1-encoding nucleic acid.

In an aspect of the invention, the inhibitory nucleic acid molecule comprises an antisense oligonucleotide complementary to a PD1-encoding nucleic acid.

In an aspect of the invention, the inhibitory protein or anti-PD-1 scFv is constitutively expressed.

In an aspect of the invention, the antigen is HPV E6 or E7.

The present invention further provides a nucleic acid comprising (a) a nucleic acid encoding genetically engineered antigen receptor that specifically binds to an antigen from HPV; and (b) an inhibitory nucleic acid molecule that reduces, or is capable of effecting reduction of, expression of a tumor target. In an aspect, the antigen is a HPV E6 or E7

In an aspect of the invention, the inhibitory protein blocks Programmed Cell Death Protein 1 (PD-1), wherein the protein is a single chain antibody (scFv).

The anti-PD-1 scFv antibody comprises following motif sequences: a heavy chain CDR1 comprising amino acids having the sequence set forth in SEQ ID NO:1; a heavy chain CDR2 comprising amino acids having the sequence set forth in SEQ ID NO:2; a heavy chain CDR3 comprising amino acids having the sequence set forth in SEQ ID NO:3; a light chain CDR1 comprising amino acids having the sequence set forth in SEQ ID NO:4; a light chain CDR2 comprising amino acids having the sequence set forth in SEQ ID NO:5; and a light chain CDR3 comprising amino acids having the sequence set forth in SEQ ID NO:6.

The present invention further provides a vector comprising the supra mentioned nucleic acid comprising (a) a nucleic acid encoding genetically engineered antigen receptor that specifically binds to an antigen from HPV; and (b) a nucleic acid molecule encoding a protein that reduces the expression of an inhibitory receptor in a tumor, wherein the vector is preferably a retroviral vector. The tumor further comprises lymphocytes or tumor-infiltrated lymphocytes. The tumor-infiltrated lymphocyts comprise inhibitory receptors.

In an aspect of the invention, a method of producing a genetically engineered T cell is provided, wherein the method comprises introducing a vector into a population of cells comprising T cells, the vector comprising a) a nucleic acid encoding genetically engineered antigen receptor that specifically binds to a first antigen, (b) a nucleic acid molecule encoding an inhibitory protein capable of leading to a reduction of expression of PD-1 or PD-L1 and/or inhibiting upregulation of PD-1 or PD-L1 in T cells in the population upon incubation under one or more conditions. In some embodiments, the first engineered antigen receptor specifically target to E6 receptor of HPV.

In an aspect of the invention, a pharmaceutical composition comprising the supra mentioned engineered T cells and a pharmaceutically acceptable carrier is provided. Also, a method for treating cancer comprising administering to a subject in need thereof, a therapeutically effective amount of the pharmaceutical composition is provided, wherein the cancer is a cervical cancer or head and neck cancer.

### BRIEF DESCRIPTION OF DRAWINGS

Exemplary embodiments are illustrated in referenced figures. It is intended that the embodiments and figures disclosed herein are to be considered illustrative rather than restrictive.

FIG. 1 is a schematic representation of a nucleic acid construct containing three genes linked by a P2A and T2A sequence: (a) the variable region of the alpha chain of a human anti-E6 TCR fused to the constant region of the TCR alpha chain; (b) the variable region of the beta chain of same human anti-E6 TCR fused to the constant region of the TCR beta chain; (c) the variable regions of the heavy and light chain of an anti-PD-1 antibody, linked with a GS linker.

FIG. 2 shows the CDR sequences of the anti-PD1 antibody sequence (c).

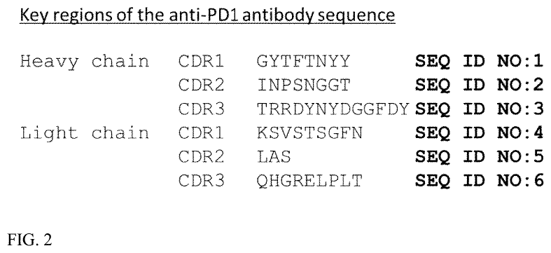

FIG. 3 shows in-vitro expression of secreted anti-PD-1 scFv in the cell culture supernatant derived from engineered T cells of the present invention.

FIG. 4. shows in-vitro expression anti-E6 TCR on engineered human T cells of the present invention.

FIG. 5. shows the binding activity of secreted anti-PD-1 scFv to PD-1 over-expressed on cell surface.

FIG. 6. shows the competitive binding activity of secreted anti-PD-1 scFv against rhPD-L1 to PD-1 over-expressed on cell surface.

FIG. 7. shows effects of secreted anti-PD-1 scFv on PD-L1-mediated inhibition of IFNγ production.

FIG. 8. shows effects of secreting anti-PD-1 scFv on IFNγ production of TCR-T cells upon antigen-specific stimulation.

FIG. 9. shows cytotoxicity of TCR-T cells against target cells.

FIG. 10 shows proliferation of TCR-T cells upon antigen-specific stimulation.

FIG. 11 shows expression of PD-1 on various TCR-T cells upon antigen-specific stimulation.

### DETAILED DESCRIPTION OF INVENTION

The following embodiments and aspects thereof are described and illustrated in conjunction with systems, compositions and methods which are meant to be exemplary and illustrative, not limiting in scope.

### Definitions

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

“Single chain variable fragment”, “single-chain antibody variable fragments” or “scFv” antibodies as used herein refers to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chains may be in any order, for example, VH-linker-VL or VL-linker-VH, so long as the specificity of the scFv to the target antigen is retained.

As used herein, the term “antigen” refers to a molecule capable of being bound by an antibody or a T cell receptor (TCR) if presented by MHC molecules. The term “antigen”, as used herein, also encompasses T-cell epitopes which are recognised by T-cell receptors. This recognition causes activation of T-cells and subsequent effector mechanisms such as proliferation of the T-cells, cytokine secretion etc. An antigen is additionally capable of being recognized by the immune system and/or capable of inducing a humoral immune response and/or a cellular immune response leading to the activation of B-lymphocytes and/or T-lymphocytes.

As used herein, the term “HPV antigen” refers to a polypeptide molecule derived from Human Papilloma Virus (HPV), preferably wherein the HPV is selected from HPV1, HPV2, HPV3, HPV4, HPV6, HPV10, HPV11, HPV16, HPV18, HPV26, HPV27, HPV28, HPV29, HPV30, HPV31, HPV33, HPV34, HPV35, HPV39, HPV40, HPV41, HPV42, HPV43, HPV45, HPV49, HPV51, HPV52, HPV54, HPV55, HPV56, HPV57, HPV58, HPV59, HPV68, HPV69. More preferably, the HPV is selected from high risk HPVs, for example, HPV16, HPV18, HPV31, HPV33, HPV35, HPV39, HPV45, HPV51, HPV52, HPV56, HPV58, HPV59, HPV68, HPV69. The HPV polypeptide molecule is selected from E6 and E7.

As used herein, the term “peripheral blood cell subtypes” refers to cell types normally found in the peripheral blood including, but is not limited to, eosinophils, neutrophils, T cells, monocytes, K cells, granulocytes, and B cells.

As used herein, the term “T cell” includes CD4+ T cells and CD8+ T cells. The term T cell also includes both T helper 1 type T cells and T helper 2 type T cells. T cells express a cell surface receptor that recognizes a specific antigenic moiety on the surface of a target cell. The cell surface receptor may be a wild type or recombinant T cell receptor (TCR), a chimeric antigen receptor (CAR), or any other surface receptor capable of recognizing an antigenic moiety that is associated with the target cell. Typically, a TCR has two protein chains (alpha- and beta-chain), which bind with specific peptides presented by an MHC protein on the surface of certain cells. TCRs recognize peptides in the context of MHC molecules expressed on the surface of a target cell. TCRs also recognize cancer antigens presented directly on the surface of cancer cells.

“Genetically modified cells”, “redirected cells”, “engineered cells”, “genetically engineered cells” or “modified cells” as used herein refer to cells that express the genetically engineered antigen receptors and checkpoint inhibitors. In some embodiments, the genetically modified cells comprise vectors that encode a genetically engineered TCR and vectors that encode one or more checkpoint inhibitors. In some embodiments, the genetically modified cells comprise a vector that encodes a genetically engineered TCR and one or more checkpoint inhibitors. In one embodiment, the genetically modified cell is a T-lymphocyte cell (T-cell). In one embodiment, the genetically modified cell is a Natural Killer (NK) cells.

As used herein, the term “genetically engineered” or “genetically modified” refers to a modification of a nucleic acid sequence of a cell, including, but not limited to, deleting a coding or non-coding region or a portion thereof or inserting a coding region or a portion thereof.

As used herein, the term “vector”, “cloning vector” and “expression vector” refers to a vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc. Most popular type of vector is a “plasmid”, which refers to a closed circular double stranded DNA loop into which additional DNA segments comprising gene of interest may be ligated. Another type of vector is a viral vector, in which a nucleic acid construct to be transported is ligated into the viral genome. Viral vectors are capable of autonomous replication in a host cell into which they are introduced or may integrate themselves into the genome of a host cell and thereby are replicated along with the host genome. Moreover, certain vectors are capable of directing the expression of genes to which they are operatively linked. Such vectors are referred to herein as “recombinant expression vectors” or simply “expression vectors”. It may be noted that the invention is intended to include such other forms of expression vectors, such as viral vectors (e.g., replication defective retroviruses, adenoviruses and adeno-associated viruses), which serve equivalent functions.

As used herein, the term “retroviral vector” and “recombinant retroviral vector” refers to a nucleic acid construct which carries, and within certain embodiments, is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms, both of which forms are included in the term “retroviral vector” and “recombinant retroviral vector”. The term “retroviral vector” and “recombinant retroviral vector” also encompass the DNA form which contains a recombinant DNA fragment and the RNA form containing a recombinant RNA fragment. The vectors may include at least one transcriptional promoter/enhancer, or other elements which control gene expression. Such vectors may also include a packaging signal, long terminal repeats (LTRs) or portion thereof, and positive and negative strand primer binding sites appropriate to the retrovirus used (if these are not already present in the retroviral vector). Optionally, the vectors may also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. By way of example, such vectors may include a 5′ LTR, a leading sequence, a tRNA binding site, a packaging signal, an origin of second strand DNA synthesis, and a 3′ LTR or a portion thereof.

“Linker” (L) or “linker domain” or “linker region” as used herein refer to an oligo- or polypeptide region from about 1 to 100 amino acids in length, which links together any of the domains/regions of the CAR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other embodiments, the linker sequences may comprise Asp-Val/Ile-Glu-X-Asn-Pro-Gly(2A)-Pro(2B) motif, which results in cleavage between the 2A glycine and the 2B proline. Other linkers will be apparent to those of skill in the art and may be used in connection with alternate embodiments of the invention.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

The term “control” refers to any reference standard suitable to provide a comparison to the expression products in the test sample.

As used herein, the term “inhibit” refers to any decrease in, for example a particular action, function, or interaction. For example, a biological function, such as the function of a protein and/or binding of one protein to another, is inhibited if it is decreased as compared to a reference state, such as a control like a wild-type state or a state in the absence of an applied agent. For example, the binding of a PD-1 protein to one or more of its ligands, such as PD-L1 and/or PD-L2, and/or resulting PD-1 signaling and immune effects is inhibited or deficient if the binding, signaling, and other immune effects are decreased due to contact with an agent, such as an anti-PD-1 antibody, in comparison to when the PD-1 protein is not contacted with the agent. Such inhibition or deficiency can be induced, such as by application of agent at a particular time and/or place, or can be constitutive, such as by continual administration. Such inhibition or deficiency can also be partial or complete (e.g., essentially no measurable activity in comparison to a reference state, such as a control like a wild-type state). Essentially complete inhibition or deficiency is referred to as blocked.

“Conditions” and “disease conditions,” as used herein may include, cancers, tumors or infectious diseases. In exemplary embodiments, the conditions include but are in no way limited to any form of malignant neoplastic cell proliferative disorders or diseases. In exemplary embodiments, conditions include any one or more of kidney cancer, melanoma, prostate cancer, breast cancer, glioblastoma, lung cancer, colon cancer, or bladder cancer.

“Cancer” and “cancerous” refers to or describe the physiological condition in mammals that is typically characterized by unregulated cell growth. The term “cancer” is meant to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of the cancers. Treatment of metastatic cancers, e.g., metastatic cancers that express PD-L1 (Iwai et al. (2005) Int. Immunol. 17:133-144) can be effected using the antibody molecules described herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In accordance with various embodiments described herein, the present invention provides engineered cells and compositions/formulations containing the engineered cells. The present invention also provides methods or processes for manufacturing the engineered cells, which may be useful for treating patients with a pathological disease or condition.

Further, in accordance with various embodiments described herein, the present invention provides a recombinant vector comprising a nucleic acid construct suitable for genetically modifying a cell, which may be used for treatment of pathological disease or condition.

Furthermore, in accordance with various embodiments described herein, the present invention provides an engineered cell comprising a nucleic acid construct suitable for genetically modifying a cell, which may be used for treatment of pathological disease or condition, wherein the nucleic acid encodes: (a) a genetically engineered antigen receptor that specifically binds to an antigen; and (b) an inhibitory protein that reduces, or is capable of effecting reduction of, expression of a tumor target. In various embodiments, the cell expresses the genetically engineered antigen receptor and the inhibitory protein. In various embodiments, the inhibitory protein is constitutively expressed.

Among the diseases, conditions, and disorders for treatment with the provided cells, compositions, methods and uses are tumors, including solid tumors, hematologic malignancies, and melanomas, and infectious diseases, such as infection with a virus or other pathogen, e.g., HPV, HIV, HCV, HBV, EBV, HTLV-1, CMV, adenovirus, BK polyomarvirus, HHV-8, MCV or other pathogens, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the uterine cervix, colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

Engineered Cells

In various embodiments, the cell that is engineered may be obtained from bacteria, fungi, humans, rats, mice, rabbits, monkeys, pig or any other species. Preferably, the cell is from humans, rats or mice. More preferably, the cell is obtained from humans. In various embodiments, the cell that is engineered is a blood cell. Preferably, the cell is a leukocyte, lymphocyte or any other suitable blood cell type. Preferably, the cell is a peripheral blood cell. More preferably, the cell is a T cell, B cell or NK cell.

In preferred embodiments, the cell is a T cell. Examples of the T cell used in the present invention include, but are not limited to: cell obtained by in vitro culture of T cells (e.g., tumor infiltrating lymphocytes) isolated from patient(s); TCR gene-modified T cells obtained by transducing T cells, isolated from the peripheral blood of patient(s), with a viral vector; and CAR-transduced T cells. Preferably, the T cell is a TCR gene-modified T cell.

In an embodiment of the invention, the cell is a NK cell.

Recombinant Vectors

Any vector or vector type may be used to deliver genetic material to the cell for example but not limited to, plasmid vectors, viral vectors, BACs, YACs, HACs. Accordingly, viral vectors that may be used include, but not limited to, are recombinant retroviral vectors, recombinant lentiviral vectors, recombinant adenoviral vectors, foamy virus vectors, recombinant adeno-associated viral (AAV) vectors, hybrid vectors and/or plasmid transposons (for example sleeping beauty transposon system) or integrase based vector systems. Other vectors that may be used in connection with alternate embodiments of the invention will be apparent to those of skill in the art.

In preferred embodiments, the vector used is a recombinant retroviral vector. The viral vector may be grown in a culture medium specific for viral vector manufacturing. Any suitable growth media and/or supplements for growing viral vectors may be used in accordance with the embodiments described herein.

Genetically Engineered Antigen Receptor

The antigen receptor that is genetically engineered is selected from but not limited to T cell receptors (TCRs), Killer-cell immunoglobulin-like receptor family (KIRs), C-type lectin receptor family, Leukocyte immunoglobulin-like receptor family (LILRs), Type 1 cytokine receptors, Type 2 cytokine receptor family, Tumor necrosis factor family, TGFβ receptor family, chemokine receptors, IgSF.

In an embodiment of the invention, the genetically engineered antigen receptor encoded by the nucleic acid construct is a genetically engineered NK cell receptor. In some embodiments, the NK cell receptor belongs to Killer-cell immunoglobulin-like receptor family (KIRs). In alternate embodiments, the NK cell receptor belongs to C-type lectin receptor family.

In preferred embodiments, the genetically engineered antigen receptor encoded by the nucleic acid construct is a genetically engineered T cell receptor (TCR). Preferably, T cell expressing this receptor is an αβ-T cell. In alternate embodiments, the T cell expressing this receptor is a γδ-T cell.

Antigens Targeted

In some embodiments, the antigen associated with the disease or disorder is selected from the group consisting of molecules expressed by HPV, HIV, HCV, HBV, EBV, HTLV-1, CMV, adenovirus, BK polyomarvirus, HHV-8, MCV or other pathogens, orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules.

Preferably, the genetically engineered antigen receptor binds to antigens from Human papillomavirus (HPV). The sub-type of HPV is selected from but not limited to, HPV1, HPV2, HPV3, HPV4, HPV6, HPV10, HPV11, HPV16, HPV18, HPV26, HPV27, HPV28, HPV29, HPV30, HPV31, HPV33, HPV34, HPV35, HPV39, HPV40, HPV41, HPV42, HPV43, HPV45, HPV49, HPV51, HPV52, HPV54, HPV55, HPV56, HPV57, HPV58, HPV59, HPV68, HPV69. In some embodiments, the sub-type of HPV targeted by the genetically engineered antigen receptor is selected from at least one high-risk HPV, for example but not limited to HPV16, HPV18, HPV31, HPV33, HPV35, HPV39, HPV45, HPV51, HPV52, HPV56, HPV58, HPV59, HPV68, HPV69.

In some embodiments, the HPV antigen is selected from but not limited to, E1, E2, E3, E4, E6 and E7, L1 and L2 proteins. In preferred embodiments, the antigen is an E6 antigen. In another preferred embodiment, the antigen is an E7 antigen. In a more preferred embodiment, the antigen is an HPV16 E6 antigen.

Accordingly, the disease or condition treated is an infectious disease or condition, such as, but not limited to, viral, retroviral, bacterial, and protozoal infections, immunodeficiency, Human Papilloma Virus (HPV), Cytomegalovirus (CMV), Epstein-Barr virus (EBV), adenovirus, BK polyomavirus. In some embodiments, the disease or condition is a viral associated malignancy for example, but not limited to, HPV, HCV, EBV, HIV, HHV-8, HTLV-1, MCV. Preferably, the viral associated malignancy for treatment with the provided compositions, cells, methods and uses is a HPV associated cancer. More preferably, the provided compositions, cells, methods can be used for treatment of solid tumors caused by a HPV associated cancer. Specifically, the diseases or conditions include HPV associated cancers, for example, but not limited to, cancer of uterine cervix, oropharynx, anus, anal canal, anorectum, vagina, vulva, and penis. More specifically, the diseases or conditions include HPV associated head and neck cancers, HPV associated cancer of uterine cervix.

Checkpoint Inhibitors

In various embodiments, the engineered cell expresses at least one checkpoint inhibitor (CPI). The inhibitory protein or CPI expressed by the engineered cells of the present invention inhibits or blocks an immune checkpoint, wherein the immune checkpoint is selected from group consisting of, but not limited to, PD-1, PD-L1, PD-L2, 2B4 (CD244), 4-IBB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, KIR family receptors, LAG-3, OX-40, PIR-B, SIRPalpha (CD47), TFM-4, TIGIT, TIM-1, TIM-3, TIM-4, VISTA and combinations thereof.

In preferred embodiments, the inhibitory protein blocks PD-1 or PD-L1. In various embodiments, the inhibitory protein is an anti-PD-1 scFv. The inhibitory protein is capable of leading to a reduction of expression of PD-1 or PD-L1 and/or inhibiting upregulation of PD-1 or PD-L1 in T cells in the population. Preferably, the inhibitory protein blocks PD-1.

Nucleic Acid Construct

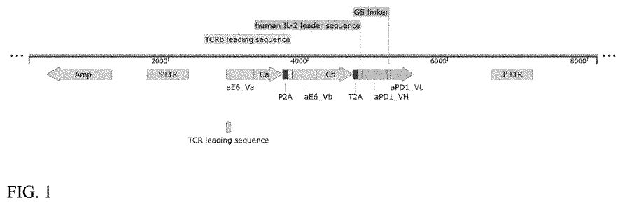

Referring to FIG. 1, according to various preferred embodiments, the nucleic acid construct includes three sequences. Preferably, the three sequences include: (a) the variable region of the alpha chain of an anti-E6 TCR fused to the constant region of the TCR alpha chain identified as “aE6_Va-Ca”, wherein aE6_Va corresponds to the variable region of the alpha chain of an anti-E6 TCR and Ca corresponds to the constant region of the TCR alpha chain; (b) the variable region of the beta chain of same anti-E6 TCR fused to the constant region of the TCR beta chain identified as “aE6_Vb-Cb”, wherein aE6_Vb corresponds to the variable region of the beta chain of same human anti-E6 TCR and Cb corresponds to the constant region of the TCR beta chain; and, (c) the variable region of the heavy chain of an anti-PD-1 antibody identified as “aPD1_VH” and the variable region of the light chain of an anti-PD-1 antibody identified as “aPD1_VL”, wherein the key regions of the anti-PD-1 antibody sequence comprise: a framework FR1 region of the heavy chain variable region; a heavy chain CDR1 comprising amino acids having the sequence set forth in SEQ ID NO:1; a framework FR2 region of the heavy chain variable region; a heavy chain CDR2 comprising amino acids having the sequence set forth in SEQ ID NO:2; a framework FR3 region of the heavy chain variable region; a heavy chain CDR3 comprising amino acids having the sequence set forth in SEQ ID NO:3; a framework FR4 region of the heavy chain variable region; a framework FR1 region of the light chain variable region; a light chain CDR1 comprising amino acids having the sequence set forth in SEQ ID NO:4; a framework FR2 region of the light chain variable region; a light chain CDR2 comprising amino acids having the sequence set forth in SEQ ID NO:5; a framework FR3 region of the light chain variable region; a light chain CDR3 comprising amino acids having the sequence set forth in SEQ ID NO:6; and a framework FR4 region of the light chain variable region.

In various embodiments, the inhibitory nucleic acid encoding for inhibitory protein comprises a sequence complementary to a PD1-encoding nucleic acid. In some embodiments, the inhibitory nucleic acid encoding for inhibitory protein comprises an antisense oligonucleotide complementary to a PD1-encoding nucleic acid.

The nucleic acid construct further comprises P2A and T2A sequences linking the supra mentioned sequences (a), (b) and, (c). Further, the variable regions of the heavy and light chain of the anti-PD-1 antibody (identified as aPD1_VH and aPD1_VL respectively) are linked with a GS linker.

The nucleic acid construct may further include other sequences which may assist and/or enable in the transfection, transduction, integration, replication, transcription, translation, expression and/or stabilization of the construct.

Method for Preparation of Engineered Cells

The present invention provides a method or process for manufacturing and using the engineered cells for treatment of pathological diseases or conditions. The method comprises the steps of: (I) isolating the T cells from a patient's blood; (II) transducing the population T cells with a viral vector including the nucleic acid construct encoding a genetically engineered antigen receptor and an inhibitory protein; (III) expanding the transduced cells in vitro; and, (IV) infusing the expanded cells into the patient, where the engineered T cells will seek and destroy antigen positive tumor cells. At the same time, these engineered T cells will block PD-1/PD-L1 immunosuppression and strengthen the antitumor immune response.

The method further comprises: transfection of T cells with the viral vector containing the nucleic acid construct of the present invention, prior to step (II).

The transfection of T cells may be achieved using any of standard methods such as calcium phosphate method, electroporation, liposomal mediated transfer, microinjection, biolistic particle delivery system, or any other known methods. In some embodiments, transfection of T cells is performed using calcium phosphate method.

According to various embodiments described herein, the present invention provides Immunotherapy for HPV associated cancers particularly HPV16 E6+ or HPV16 E7+ cancers. The engineered T cells recognize tumor antigen HPV E6 and simultaneously secrete a single-chain antibody (scFv) that blocks Programmed Cell Death Protein 1 (PD-1). These engineered T cells demonstrate stronger antitumor response and reduced T cell exhaustion.

It has been found experimentally that the PD-1 checkpoint blockade is more effective with this invention because (1) anti-PD-1 drug delivery is localized to the tumor site and (2) the anti-PD-1 single-chain antibody binds more strongly than currently existing antibodies. Also, toxicity due to non-specific inflammation is reduced because anti-PD-1 drug delivery is localized to the tumor site. It has been found that the combination of anti-E6 TCR and anti-PD-1 improves T cell activation and/or prevent T cell exhaustion compared to existing alternatives.

Also, the present invention may be used to create a personalized anti-tumor immunotherapy. Anti-E6+/anti-PD-1 engineered T cells can be easily produced from a patient's blood. These engineered T cells are then reinfused into the patient as a cellular therapy product. This product could be applied to any patient who has an HPVE6+ tumor, including cervical cancer, head and neck cancer and, others.

Compositions, Formulations and Methods of Administration

The present invention provides compositions (including pharmaceutical and therapeutic compositions) containing the engineered T cells and populations thereof, produced by the disclosed methods. Also provided are methods, e.g., therapeutic methods for administrating the engineered T cells and compositions thereof to subjects, e.g., patients.

A. Compositions and Formulations

Compositions including the engineered T cells for administration, including pharmaceutical compositions and formulations, such as unit dose form compositions including the number of cells for administration in a given dose or fraction thereof are provided. The pharmaceutical compositions and formulations may include one or more optional pharmaceutically acceptable carrier or excipient. In some embodiments, the composition includes at least one additional therapeutic agent.

In some embodiments, the choice of carrier is determined in part by the particular cell (e.g., T cell or NK cell) and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents used in the invention includes, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for a particular indication, disease, or condition being treated with the engineered T cells, preferably those with activities complementary to the cells, where the respective activities do not adversely affect one another. Such active ingredients are suitably present in combination in amounts that are effective for the purpose intended. Thus, in some embodiments, the pharmaceutical composition may further include other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

The compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

B. Methods of Administration and Uses of Engineered T Cells in Adoptive Cell Therapy

Provided are methods of administering the cells, populations, and compositions, and uses of such cells, populations, and compositions to treat or prevent diseases, conditions, and disorders, including cancers. In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by the engineered T cells.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 104 and at or about 109 cells/kilograms (kg) body weight, such as between 105 and 106 cells/kg body weight, for example, at least or at least about or at or about 1×105 cells/kg, 1.5×105 cells/kg, 2×105 cells/kg, or 1×106 cells/kg body weight. For example, in some embodiments, the cells are administered at, or within a certain range of error of, between at or about 104 and at or about 109 T cells/kilograms (kg) body weight, such as between 105 and 106 T cells/kg body weight, for example, at least or at least about or at or about 1×105 T cells/kg, 1.5×105 T cells/kg, 2×105 T cells/kg, or 1×106 T cells/kg body weight.

In some embodiments, the cells are administered at or within a certain range of error of between at or about 104 and at or about 109 CD4+ and/or CD8+ cells/kilograms (kg) body weight, such as between 105 and 106 CD4+ and/or CD8+ cells/kg body weight, for example, at least or at least about or at or about 1×105 CD4+ and/or CD8+ cells/kg, 1.5×105 CD4+ and/or CD8+ cells/kg, 2×105 CD4+ and/or CD8+ cells/kg, or 1×106 CD4+ and/or CD8+ cells/kg body weight.

In some embodiments, the cells are administered at or within a certain range of error of, greater than, and/or at least about 1×106, about 2.5×106, about 5×106, about 7.5×106, or about 9×106 CD4+ cells, and/or at least about 1×106, about 2.5×106, about 5×106, about 7.5×106, or about 9×106 CD8+ cells, and/or at least about 1×106, about 2.5×106, about 5×106, about 7.5×106, or about 9×106 T cells. In some embodiments, the cells are administered at or within a certain range of error of between about 108 and 1012 or between about 1010 and 1011 T cells, between about 108 and 1012 or between about 1010 and 1011 CD4+ cells, and/or between about 108 and 1012 or between about 1010 and 1011 CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 111 (1995), and U.S. Pat. No. 5,087,616.

C. Dosing Schedule or Regimen

In some embodiments, repeated dosage methods are provided in which a first dose of cells is given followed by one or more second consecutive doses. The timing and size of the multiple doses of cells generally are designed to increase the efficacy and/or activity and/or function of TCR-expressing engineered T cells, when administered to a subject in adoptive therapy methods. In some embodiments, the repeated dosings reduce the downregulation or inhibiting activity that can occur when inhibitory immune molecules, such as PD-1 and/or PD-L1 are upregulated on TCR-expressing engineered T cells. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first or consecutive dose is a single or continuous administration of the specified number of cells, given or initiated at a single point in time. In some contexts, however, the first or consecutive dose is administered in multiple injections or infusions over a period of no more than three days, such as once a day for three days or for two days or by multiple infusions over a single day period.

Thus, in some aspects, the cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses may be administered in a plurality of compositions over a period of no more than 3 days.

The term “split dose” refers to a dose that is split so that it is administered over more than one day. This type of dosing is encompassed by the present methods and is considered to be a single dose.

Thus, the first dose and/or consecutive dose(s) in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the first dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject received a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses, with each consecutive dose given within a period of time in which an inhibitory immune molecule, such as PD-1 and/or PD-L1, has been upregulated in cells in the subject from an administered first dose. It is within the level of a skilled artisan to empirically determine when to provide a consecutive dose, such as by assessing levels of PD-1 and/or PD-L1 in antigen-expressing, such as CAR-expressing cells, from peripheral blood or other bodily fluid.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×106 cells/kg body weight of the subject and 5×106 cells/kg, between about 0.75×106 cells/kg and 3×106 cells/kg or between about 1×106 cells/kg and 2×106 cells/kg, each inclusive.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, the receptor, e.g., the TCR, expressed by the cells in the consecutive dose contains at least one immunoreactive epitope as the receptor, e.g., the TCR, expressed by the cells of the first dose. In some embodiments, the receptor, e.g., the TCR, expressed by the cells administered in the consecutive dose is identical to the receptor, e.g., the TCR, expressed by the first dose or is substantially identical to the receptor, e.g., the TCR, expressed by the cells of administered in the first dose.

The receptors, such as TCRs, expressed by the cells administered to the subject in the various doses generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells in the first dose express a CAR that specifically binds to an antigen expressed.

### WORKING EXAMPLES

The following examples are not intended to limit the scope of the claims to the invention, but is rather intended to be exemplary of certain embodiments. Any variations in the exemplified methods which occur to the skilled artisan are intended to fall within the scope of the present invention.

Construct Design.

An MP71 retroviral vector construct containing three coding regions was generated using standard molecular biology techniques, wherein the three coding regions were: (A) the variable region of the alpha chain of a human anti-E6 TCR fused to the constant region of the TCR alpha chain (designated as aE6_Va-Ca); (B) the variable region of the beta chain of same human anti-E6 TCR fused to the constant region of the TCR beta chain (designated as aE6_Vb-Cb); and, (C) the variable regions of the heavy (designated as aPD1_VH) and light chain (designated as aPD1_VL) of a novel anti-PD-1 antibody, linked with a GS linker. The retroviral vector obtained is generally designated as E6.αPD1_m11. The schematic representation of the retroviral vector construct used in this study is shown in FIG. 1.

Cell Lines and Media.

HEK-293T and CaSki cells were purchased from ATCC. Peripheral blood mononuclear cells (PBMCs) from anonymous donors were purchased from Hemacare. 293T-PD-1 cells were produced by lentiviral transduction of 293T cells with a vector overexpressing human PD-1. Cells were cultured in DMEM+10% FBS, RPMI+10% FBS, or X-Vivo+5% human serum A/B+1% HEPES+1% GlutaMAX.

Retroviral Vector Production.

Retroviral vectors were prepared by transient transfection of 293T cells using a standard calcium phosphate precipitation protocol. Viral supernatants were harvested at 48 h and used to transduce T cells.

T Cell Transduction and Expansion.

Before retroviral transduction, PBMCs were activated for 2 days by culturing with T cell activator beads and human IL-2. For transduction, freshly harvested retroviral supernatant was spin-loaded onto non-tissue culture-treated 24-well plates coated with 15 μg RetroNectin per/well (Clontech Laboratories) by centrifuging 2 hr at 2,000 g at 32 C. Activated PBMCs were loaded onto the plates and spun at 600 g at 32 C for 30 min. T cells were incubated at 37 C and 5% CO2. Culture medium was replenished every 2 days.

TCR and PD-1 Staining.

All antibodies were purchased from Biolegend. Expression of the recombinant TCR was detected 72 h after transfection by antibody staining to TCR beta chain followed by flow cytometry. Expression of PD-1 was detected 72 h after co-culture with CaSki target cells by antibody staining to PD-1. CD3, CD4, and CD8 staining was performed simultaneously.

In Vitro Anti-PD-1 scFv Expression.

293T cells were transfected with retroviral vectors encoding either E6, E6.αPD1_m11 or E6.αPD1_5C4 TCR transgenes. The cell culture supernatant was then collected 48 hrs post-transfection. The anti-PD-1 scFv in 20 μl of supernatant was detected by ELISA.

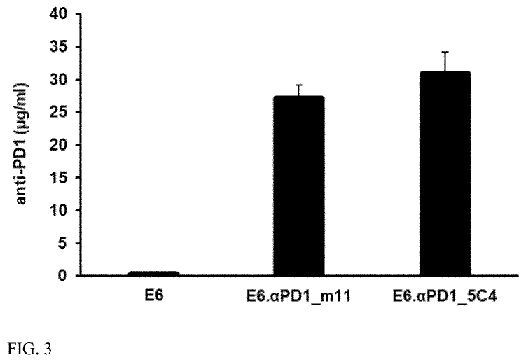

Results: FIG. 3 shows the expression of secreted anti-PD-1 scFv in the cell culture supernatant. E6 designates E6 TCR with no anti-PD-1; E6.αPD1_m11 designates E6 TCR with novel anti-PD-1 single-chain antibody of the present invention; E6.αPD1_5C4 designates E6 TCR with control anti-PD-1 single-chain antibody derived from a published sequence.

In Vitro Anti-E6 TCR-T Expression.

Primary T cells were transduced with the indicated constructs. After 72 hours of culture, expression of the recombinant TCR was detected by antibody staining to TCR beta chain. A viable CD3+ lymphocyte gating strategy was used.

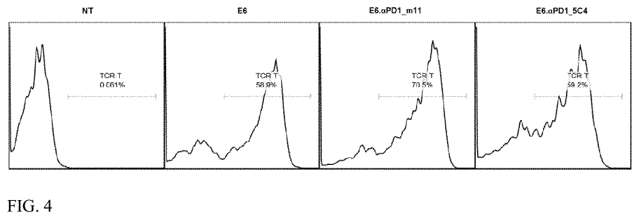

Results: FIG. 4 shows a panel wherein the anti-E6 TCR is expressed strongly in T cells containing the E6.αPD1_m11 construct.

Binding Activity of Secreted Anti-PD-1 scFv.

293T cells were transfected with retroviral vectors encoding either E6, E6.αPD1_m11 or E6.αPD1_5C4 TCR transgenes. The cell culture supernatant was collected 48 hrs post-transfection. 293T-PD-1 cells were incubated with 300 μl of the supernatant for 30 min at room temperature and then the anti-HA tag antibody was used to stain the cells and detect the secreted HA tagged anti-PD-1 antibody bound to the 293T-PD-1 cells.

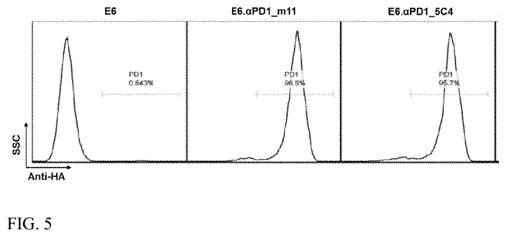

Results: As seen in FIG. 5, Both of the secreted anti-PD1 bound strongly to 293T-PD-1 cells. The E6.αPD1_m11 and E6.αPD1_5C4 antibodies have comparable binding affinities to the PD-1 expressed on the cell surface.

Competitive Binding of Recombinant PD-L1.

293T-PD1 cells were incubated with 1 μl of 100 μg/ml rhPD-L1/Fc and 300 μl of supernatant of E6, E6.αPD1_m11 or E6.αPD1_5C4 TCR-transfected 293T cell culture for 30 min. The cells were then stained with PE-conjugated anti-human Fc.

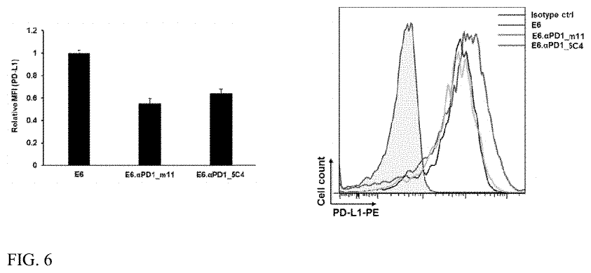

Results: As seen in FIG. 6, supernatant from E6.αPD1_m11 and E6.αPD1_5C4 TCR-T cells was able to compete with recombinant PD-L1, thus demonstrating that the single-chain anti-PD1 antibody can block the interaction between PD-1 and its ligand PD-L1.

In Vitro TCR-T IFNγ Activation.

The 96-well assay plates were coated with 3 μg/ml of anti-human CD3 antibody at 4° C. overnight. On the second day, the supernatant of the wells was aspirated and the wells were washed once with 100 μl per well of PBS. 10 μg/ml of rhPD-L1/Fc in 100 μl of PBS were added. In each well, 100 μg/ml of goat anti-human IgG Fc antibody in 10 μl of PBS were then added. The assay plate was incubated for 4 hours at 37° C. Human T cells were harvested, washed once and then resuspended to 1×10⁶ cells/ml in TCM. The wells of the assay plate were aspirated. Then, 100 μl of human T-cell suspension (1×10⁵) and 100 μl of supernatant of E6, E6.αPD1_m11 or E6.αPD1_5C4 TCR-transfected 293T cell culture 2-day post-transfection, supplemented with GolgiPlug, were added to each well. The plate was covered and incubated at 37° C. and 5% CO2 overnight. After incubation, T cells were harvested and stained with IFN-γ intracellularly.

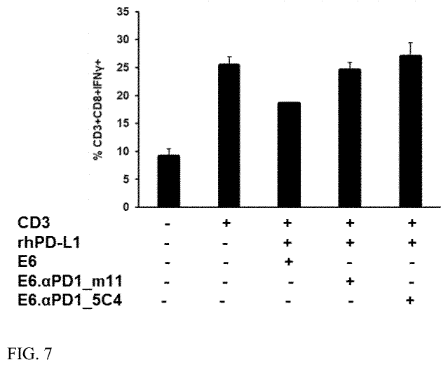

Results: Referring to FIG. 7, TCR-T cells containing the E6 TCR could be activated by CD3 antibodies, as measured by IFNγ expression, but that activation was reduced by the introduction of recombinant PD-L1 (rhPD-L1). However, for both E6.αPD1_m11 and E6.αPD1_5C4, PD-L1 did not reduce activation.

In Vitro TCR-T IFNγ Secretion.

TCR-T cells were cocultured for either 48 hrs or 72 hrs with Ca Ski cells at 1:0, 1:2, 1:1, and 3:1 effector-to-target ratios. The supernatant was then collected and the IFN-γ production was measured using a human IFN-γ ELISA kit according to the manufacturer's instructions.

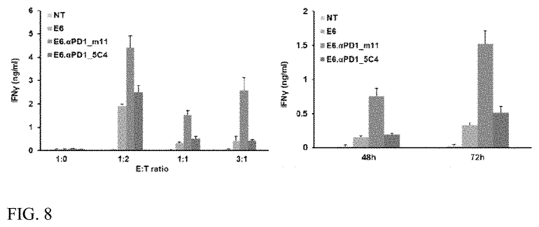

Results: The effects of secreting anti-PD-1 scFv on IFNγ production of TCR-T cells upon antigen-specific stimulation is shown in FIG. 8 (NT designates non-transduced control which was used as control). As seen from FIG. 8, IFNγ secretions were detected from the supernatant in both E6.αPD1_m11 and E6.αPD1_5C4 TCR-T cells, however, IFNγ production from E6.αPD1_m11 was significantly higher.

Specific Cell Lysis (Cytotoxicity).

Ca Ski tumor cells were pre-stained with CFSE and then cocultured for overnight with E6, E6.αPD1_m11 or E6.αPD1_5C4 TCR-T cells at 1:2, 1:1, and 3:1 effector-to-target ratios. The cytotoxicity of T cells against Ca Ski cells was measured by Annexin V/7-AAD staining. Non-target 293T cells were used as a control.

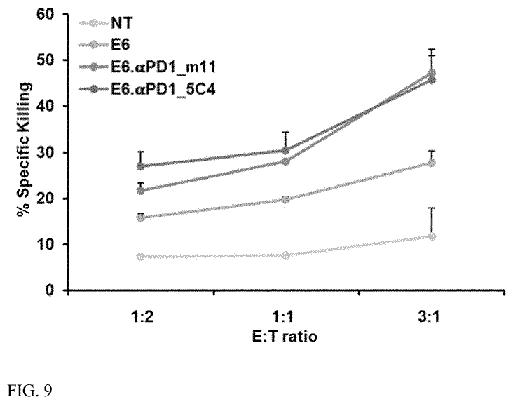

Results: As seen in FIG. 9, all E6 TCR-T cells killed E6+ target cells (CaSki) in a specific manner. E6.αPD1_m11 and E6.αPD1_5C4 killed target cells more efficiently than E6 alone.

In Vitro TCR-T Proliferation.

E6, E6.αPD1_m11 and E6.αPD1_5C4 TCR-T cells were pre-stained with CFSE. The stained T cells were then cocultured for 72 hours with Ca Ski cells and the intensity of CFSE was measured by flow cytometry. Nontransduced (NT) T cells were used as a control.

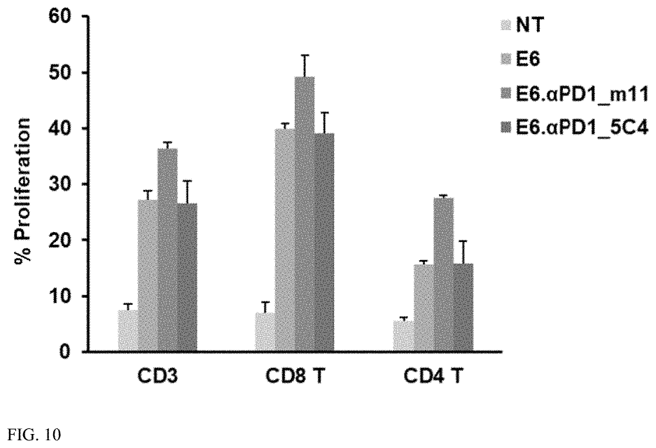

Results: As seen in FIG. 10, exposure to E6+ target cells stimulated all E6 TCR-T cells to proliferate, another measure of activation, however E6.αPD1_m11 TCR-T cells proliferated faster than the other TCR-T cells tested.

In Vitro Expression of PD-1 Upon Antigen-Specific Stimulation.

All the TCR-T cells were cocultured with Ca Ski cells for 72 hrs. The T cells were then collected and PD-1 expression on the cell surface was measured by flow cytometry. PD-1-expressing CD8 T or CD4 T cells were gated, and their percentage over total CD8+T or CD4+ T cells was shown. NT indicates nontransduced T cells, which were used as a control.

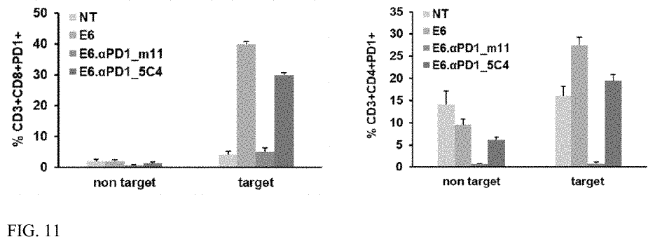

Results: As seen in FIG. 11, after antigen stimulation, the inhibitory receptor PD-1 is upregulated on T cells containing the E6 TCR and the E6.αPD1_5C4. However, PD-1 is not upregulated on cells expressing E6.αPD1_m11.

In Vivo TCR-T Efficacy.

6-8 weeks female NSG mice were subcutaneously implanted with 2e6 Ca Ski tumor cells, 12 days later, 10 ug Poly (I:C) were given to each tumor-bearing mouse via i.p. 24 hours later, 10e6 E6-TCR-T, E6.αPD1_m11-TCR-T or untransduced control PBMCs were injected in the mice via tail vein. Tumor sizes were measured twice a week to assess TCR-T anti-tumor efficacies, mouse body condition and body weight were measured twice a week to assess TCR-T associated toxicity.

Results (no data): If TCR-T cells are effective, tumor growth is expected to be slower for E6.αPD1_m11 compared to E6 and untransduced controls. In addition, T cell counts of E6.αPD1_m11 TCR-T cells may increase compared to untransduced controls.

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

1. An engineered T cell, comprising:
a nucleic acid encoding (a) genetically engineered antigen receptor that specifically binds to an antigen from HPV; and
(b) an inhibitory protein that reduces function or expression of inhibitory receptors in a tumor.

2. The engineered T cell of claim 1, wherein the antigen comprises E6 or E7.

3. The engineered T cell of claim 1, wherein tumor target comprises one or more of PD-1.

4. The engineered T cell of claim 3, wherein the inhibitory protein is an anti PD1 antibody.

5. The engineered T cell of claim 4, wherein the anti PD1 antibody is a single chain antibody.

6. The engineered T cell of claim 5, wherein the anti PD1 antibody comprised motif sequences 1) heavy chain CDR1 of GYTFTNYY, CDR2 of INPSNGGT, and CDR3 of TRRDYNYDGGFDY; 2) Light chain CDR1 of KSVSTSGFN, CDR2 of LAS and CDR3 of QHGRELPLT.

7. The engineered T cell of any of claims 1-6, wherein the inhibitory nucleic acid molecule comprises a sequence complementary to a PD1-encoding nucleic acid.

8. The engineered T cell of any of claims 1-6, wherein the inhibitory nucleic acid molecule comprises an antisense oligonucleotide complementary to a PD1-encoding nucleic acid.

9. The engineered T cell of any of claims 1-6, wherein the inhibitory protein or antibody is constitutively expressed.

10. The engineered T cell of claim 9, wherein the inhibitory protein is an antibody PD1 which is constitutively expressed.

11. A nucleic acid comprising (a) a nucleic acid encoding genetically engineered antigen receptor that specifically binds to an antigen from HPV; and (b) an inhibitory nucleic acid molecule that reduces the expression of an inhibitory receptor in a tumor.

12. The nucleic acid of claim 11, wherein the antigen receptor is E6 of HPV.

13. The nucleic acid of claim 12, wherein the tumor target is PD1.

14. Polypeptides encoded by the nucleic acid of any of claims 11-13.

15. A vector comprising the nucleic acid of any of claims 11-13.

16. The vector of claim 15, wherein vector is a retroviral vector.

17. A method of producing a genetically engineered T cell, comprising introducing a vector comprising 1) a nucleic acid encoding genetically engineered antigen receptor that specifically binds to a first antigen into a population of cells comprising T cells, the first antigen receptor specifically target to E6 receptor of HPV, (b) a nucleic acid molecule encoding an inhibitory protein capable of leading to a reduction of expression of PD-1 or PD-L1 and/or inhibiting upregulation of PD-1 or PD-L1 in T cells in the population upon incubation under one or more conditions.

18. A pharmaceutical composition, comprising the engineered T cell of any of claims 1-10 and a pharmaceutically acceptable carrier.

19. A method for treating cancer comprising administering to a subject in need thereof, a therapeutically effective amount of the pharmaceutical composition of claim 18.

20. The method of claim 19, wherein the cancer is cervical cancer or head and neck cancer.

21. The method of claim 20, further comprising administering to the subject a therapeutically effective amount of an existing therapy comprising chemotherapy or radiation.

22. The method of claim 21, wherein the cell and the existing therapy are administered sequentially or simultaneously.

23. The engineered T cell in claim 1, wherein the tumor comprises lymphocytes or tumor-infiltrating lymphocytes.

24. The nucleic acid of claim 11, wherein the tumor comprises lymphocytes or tumor-infiltrating lymphocytes.
